(19) United States
(12) Patent Application Publication
(10) Pub. No.: US 2023/0390790 A1
(43) Pub. Date: Dec. 7, 2023

(54) ELECTROSTATIC SPRAY NOZZLE FILM AND ELECTROSTATIC SPRAY SYSTEM INCLUDING SAME

(71) Applicants: JMICRO INC., Gwangmyeong-si Gyeonggi-do (KR); KOREA ADVANCED INSTITUTE OF SCIENCE AND TECHNOLOGY, Daejeon (KR); DONGSHIN UNIVERSITY INDUSTRY-ACADEMY COOPERATION, Naju-si Jeollanam-do (KR)

(72) Inventors: Seung Seob LEE, Daejeon (KR); Ji Hun JEONG, Daejeon (KR); Jin Young CHOI, Naju-si Jeollanam-do (KR); Jung Woo LEE, Daejeon (KR)

(21) Appl. No.: 18/248,950

(22) PCT Filed: Oct. 8, 2021
(86) PCT No.: PCT/KR2021/013917

§ 371 Date: Apr. 13, 2023

(30) Foreign Application Priority Data
Oct. 15, 2020 (KR) 10-2020-0133735

(51) Int. Cl. B05B 5/025; B05B 5/16; H01M 8/04119

(52) U.S. Cl. CPC B05B 5/025; B05B 5/16; H01M 8/04126

(57) ABSTRACT

Provided are an electrostatic spray nozzle film and an electrostatic spray system. The electrostatic spray nozzle film includes a first film including a first base layer and a plurality of nozzles in such a configuration that each of the plurality of nozzles protrudes outward from a surface of the first base layer, has a truncated column shape, and has a first opening therein penetrating through the first base layer; a second film including a second base layer positioned on the first film and a plurality of surrounding electrodes positioned on the second base layer and corresponding to the nozzles, respectively; and a third film positioned on the second film and having a flow path through which water for an electrostatic spray flows, wherein in a plain view, the plurality of surrounding electrodes are arranged to surround the nozzles, respectively, and a same voltage is applied to the plurality of surrounding electrodes.

### TECHNICAL FIELD

An embodiment of the present disclosure relates to an electrostatic spray nozzle film and an electrostatic spray system having the same.

### BACKGROUND ART

Electrostatic spraying is a process of dividing liquid into fine droplets by electric force and spraying the fine droplets. For example, the liquid passing through a nozzle is formed into a Taylor cone by an electromagnetic force, passes through a short liquid column section, and is broken into droplets by a repulsive force between liquid particles, so that the liquid can be sprayed in fine droplets. In this case, since the fine droplets to be sprayed are under charged state, the electrostatic spraying is used in dust collectors, etc. On the other hand, when the liquid is water, a high voltage has to be applied for the electrostatic spraying, and the water is difficult to be sprayed stably due to the high surface tension and electrical conductivity thereof.

### DISCLOSURE

### Technical Problem

Embodiments of the present disclosure provide an electrostatic spray nozzle film for stably spraying water to thereby be applied to various fields and an electrostatic spray system having the same.

### Technical Solution

An example embodiment of the present disclosure may disclose an electrostatic spray nozzle film including a first film including a first base layer and a plurality of nozzles in such a configuration that each of the nozzles may protrude outward from a surface of the first base layer with a truncated column shape and may include a first opening penetrating through the first base layer, a second film including a second base layer positioned on the first film and a plurality of surrounding electrodes positioned on the base layer and corresponding to the nozzles, respectively, and a third film positioned on the second film and having a flow path through which water for an electrostatic spray may flow. In a plan view, the plurality of surrounding electrodes may be arranged to surround the nozzles, respectively, and the same voltage may be applied to the plurality of surrounding electrodes.

### Advantageous Effects of Disclosure

According to embodiments of the present disclosure, as a constant voltage may be applied to the surrounding electrodes surrounding the corresponding nozzle, an electric field may be easily concentrated on the end portion of the nozzle, to thereby perform a stable electrostatic spray of water. In addition, as a constant voltage is applied to the surrounding electrodes, a driving voltage required for the electrostatic spray of water may be reduced, to thereby improve the efficiency of the electrostatic spray.

In addition, since the ultrapure water generated from the fuel cell of the electric power unit may be electrostatically sprayed, the efficiency of the electrostatic spray may be improved, no additional water may be required for the electrostatic spray and the nozzle clogging caused by the foreign matters, etc. in water may be prevented, to thereby improve the efficiency of the electrostatic spray.

In addition, the humidified air in the electrostatic spray unit may be supplied to the fuel cell of the electric power unit, to thereby improve the efficiency of the fuel cell.

### BRIEF DESCRIPTION OF DRAWINGS

FIG. 1 is a plan view schematically illustrating an example embodiment of an electrostatic spray nozzle film according to an embodiment of the present disclosure.

FIG. 2 is an enlarged plan view of portion A in FIG. 1.

FIG. 3 is a cross-sectional view schematically illustrating an example of a cross-section cut along line I-I′ in FIG. 2.

FIG. 4 is a cross-sectional view schematically illustrating another example of the cross section cut along I-I′ in FIG. 2.

FIG. 5 is a plan view illustrating a modified example of the electrostatic spray nozzle film shown in FIG. 1.

FIG. 6 is a cross-sectional view illustrating another modified example of the electrostatic spray nozzle film shown in FIG. 1.

FIG. 7 is a block diagram schematically showing an example embodiment of an electrostatic spray system according to an embodiment of the present disclosure.

FIG. 8 is a perspective view schematically illustrating an example embodiment of an electrostatic spray unit and an electric power unit shown in FIG. 7.

FIG. 9 is a perspective view schematically illustrating another example embodiment of the electrostatic spray unit shown in FIG. 7.

FIG. 10 is a plan view schematically showing an example embodiment of a wall portion shown in FIG. 8.

FIG. 11 is a cross-sectional view schematically illustrating an example of a cross-section cut along line II-II′ in FIG. 10.

### MODE FOR INVENTION

An example embodiment of the present disclosure may disclose an electrostatic spray nozzle film including a first film including a first base layer and a plurality of nozzles in such a configuration that each of the nozzles may protrude outward from a surface of the first base layer with a truncated column shape and may include a first opening penetrating through the first base layer, a second film including a second base layer positioned on the first film and a plurality of surrounding electrodes positioned on the base layer and corresponding to the nozzles, respectively, and a third film positioned on the second film and having a flow path through which water for an electrostatic spray may flow. In a plan view, the plurality of surrounding electrodes may be arranged to surround the nozzles, respectively, and the same voltage may be applied to the plurality of surrounding electrodes.

In an example embodiment, the second base layer may include a plurality of second openings connecting the first openings of the plurality of nozzles and the flow path with each other, and a width of each of the second openings may be greater than a width of the first opening corresponding to the second opening.

In an example embodiment, the plurality of surrounding electrodes surround the second openings, respectively.

In an example embodiment, the electrostatic spray nozzle film may further include a light-emitting device between the plurality of surrounding electrodes in a plan view.

In an example embodiment, the electrostatic spray nozzle film may further include a fourth film stacked on the third film and including at least one of an electrochromic film and an antenna film.

Another example embodiment of the present disclosure discloses an electrostatic spray system comprising an electrostatic spray unit including an electrostatic spray nozzle film, an electric power unit configured to supply power and ultrapure water to the electrostatic spray unit, and a control unit configured to control operations of the electrostatic spray unit and the electric power unit. A humidified air in the electrostatic spray unit may be supplied to a fuel cell of the electric power unit.

### MODE FOR CARRYING OUT THE INVENTION

Since the present disclosure may be modified variously and may have various embodiments, some particular embodiments will be illustrated in the drawings and described in detail in the detailed description. Effects and features of the present disclosure and methods of achieving the same will be apparent with reference to embodiments described below in detail with reference to the drawings. However, the present disclosure is not limited to the embodiments disclosed below and may be implemented in various forms.

In the following embodiments, terms such as first and second are used for the purpose of distinguishing one element from another element without limiting meaning.

In the following embodiment, the singular expression includes the plural expression unless it is explicitly meant differently in the context.

In the following embodiments, the terms include or have mean that a feature or component described in the specification exists, and the possibility of adding one or more other features or components is not excluded in advance.

In the following embodiment, when a part such as a film, a region, a component, etc. is on or above another part, it includes not only a case directly on the other part, but also a case where another film, region, component, etc. is interposed therebetween.

In the drawings, the size of components may be exaggerated or reduced for convenience of description. For example, since the size and thickness of each component shown in the drawings are arbitrarily shown for convenience of description, the present disclosure is not necessarily limited to the illustrated.

Hereinafter, embodiments of the present disclosure will be described in detail with reference to the accompanying drawings, and the same reference numerals will denote the same or corresponding components when describing with reference to the drawings.

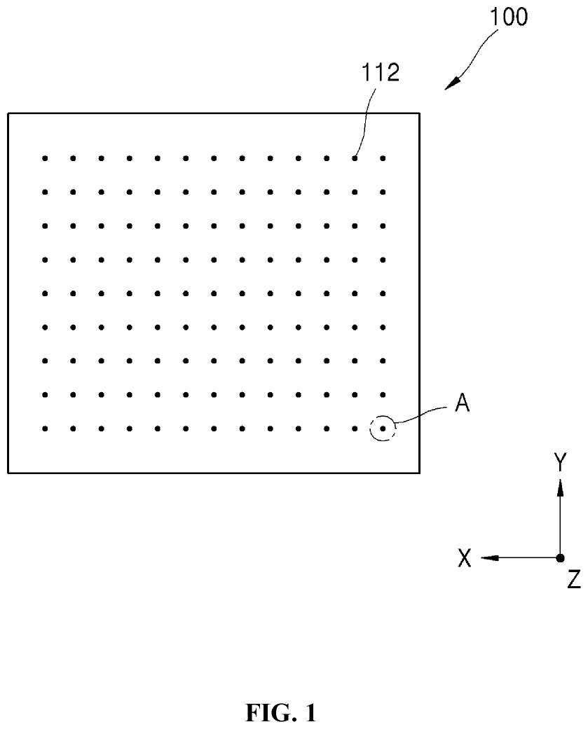

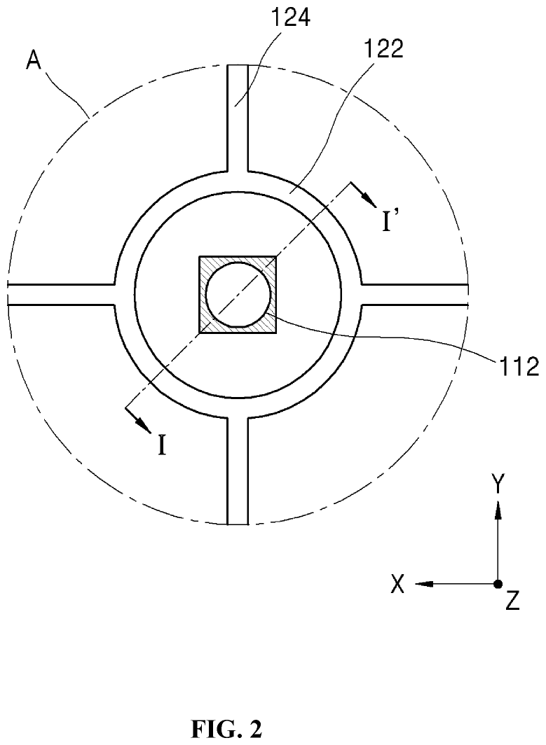

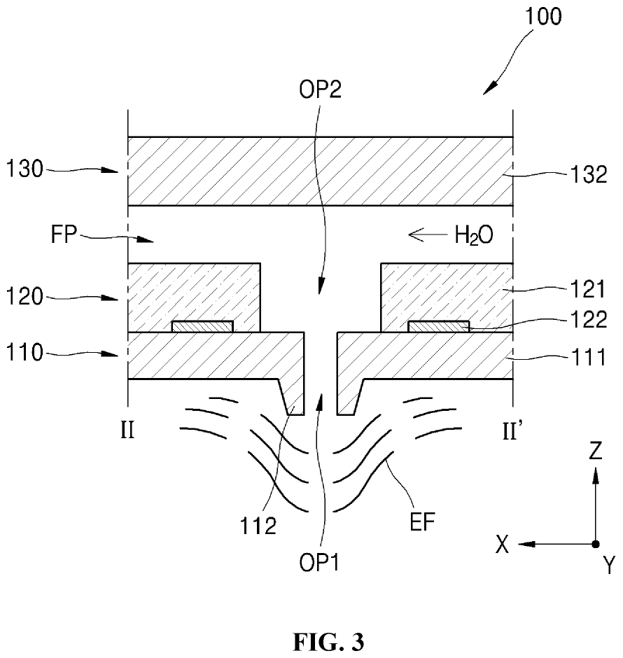

FIG. 1 is a plan view schematically illustrating an example embodiment of an electrostatic spray nozzle film according to an embodiment of the present disclosure, FIG. 2 is an enlarged plan view of portion A in FIG. 1, and FIG. 3 is a cross-sectional view schematically illustrating an example of a cross-section cut along line I-I′ in FIG. 2.

Referring to FIGS. 1 to 3, an electrostatic spray nozzle film 100 may include a first film 110, a second film 120, and a third film 130 that sequentially stacked, and the first film 110 may include a plurality of nozzles 112 for spraying water. FIG. 1 is a plan view showing a bottom surface of the electrostatic spray nozzle film 100.

Specifically, the first film 110 may include a first base layer 111 and the plurality of nozzles 112 protruding outward from the surface of the first base layer 111.

The first base layer 111 may be a transparent film. The first base layer 111 may include polyimide, acrylic, polycarbonate, polyethylene terephthalate, polyethylene, polypropylene, polysulfone, polymethyl methacrylate, triacetylcellulose, polydimethylsiloxane, etc.

As shown in FIG. 3, each of the plurality of nozzles 112 may have a shape in which a width gradually decreases in a direction protruding from the surface of the first base layer 111. Each of the plurality of nozzles 112 may have a truncated column shape such as a polygonal truncated column, a truncated cone, etc. In addition, a first opening OP1 formed in the center of the nozzle 112 may extend through the first base layer 111 with a predetermined width.

For example, the nozzle 112 may have a quadrangular truncated column shape. At this time, the base of the quadrangular truncated column shape, which is in contact with the surface of the first base layer 111, has a width of 150 μm or less, a length of 150 μm or less, and a height of 50 μm or more, and a diameter of the first opening OP1 in the nozzle 112 may be about 50 μm. As such, since each of the nozzles 112 has a micro size and a truncated column shape, a starting voltage of the electrostatic spray may be reduced and the fine droplets may be easily sprayed, so that the spraying may be performed more stably.

As shown in FIG. 1, the plurality of nozzles 112 may be arranged with being spaced apart from each other by a predetermined gap distance. For example, the gap distance between the nozzles 112 may be 1 mm to 3 mm.

The second film 120 may include a plurality of surrounding electrodes 122 corresponding to the plurality of nozzles 112, respectively. Specifically, the second film 120 may include a second base layer 121, and the plurality of surrounding electrodes 122 may be positioned on the second base layer 121.

The second base layer 121 may be a transparent film. The second base layer 121 may include polyimide, acrylic, polycarbonate, polyethylene terephthalate, polyethylene, polypropylene, polysulfone, polymethyl methacrylate, triacetylcellulose, polydimethylsiloxane, etc.

The plurality of surrounding electrodes 122 may be inserted into a surface of the second base layer 121 and positioned in the second base layer 121. For example, the surface of the second base layer 121 and the surface of the plurality of surrounding electrodes 122 may be coplanar with each other, and the plurality of surrounding electrodes 122 may be in contact with the second base layer 121 and the first base layer 111 therebetween.

As shown in FIG. 2, in a plan view, the plurality of surrounding electrodes 122 may be arranged in such a configuration that each surrounding electrode 122 surrounds the corresponding nozzle 112. For example, the surrounding electrode 112 may have a circular or donut shape with a diameter of about 2 mm. The plurality of surrounding electrodes 112 may be spaced apart from each other and electrically connected with each other by a connection wiring 124, so that the plurality of surrounding electrodes 112 may have the same electrical potential.

The plurality of surrounding electrodes 112 and the connection wiring 124 may be integrally formed. For example, a plurality of grooves may be formed on the second base layer 121 at positions where the plurality of surrounding electrodes 112 and the connection wiring 124 are to be formed, and a conductive paste may be printed in the grooves by a screen printing, to thereby form the plurality of surrounding electrodes 112 and the connection wiring 124. In another example embodiment, the plurality of surrounding electrodes 112 and the connection wiring 124 may be formed in the groove of the second base layer 121 by a deposition method or the like. At this time, a seed layer may be formed in the grooves of the second base layer 121 by the screen printing in advance so as to facilitate the deposition of the surrounding electrodes 112 and the connection wiring 124.

A constant voltage may be applied to the plurality of surrounding electrodes 112 and the connection wiring 124. As described above, when a voltage is applied to the plurality of surrounding electrodes 112, the electric field EF may be concentrated around an end portion of the nozzle 112, and thus the droplets may be sprayed more stably and the starting voltage of the electrostatic spray may be reduced, to thereby improve the spray efficiency of the electrostatic spray. For example, when no surrounding electrode 112 is provided, the voltage applied to the water may be 9 kV for performing the electrostatic spray, however, when a voltage of 5V is applied to the surrounding electrode 112, the voltage applied to the water may be reduced to 1 to 2 kV for performing the electrostatic spray.

In addition, when a voltage is applied to the plurality of surrounding electrodes 112, heat is generated by the resistance of the surrounding electrodes 112 and the temperature of the water passing through the nozzle 112 may increase. When the temperature of the water increases, the evaporation rate of the droplets may increase, which is advantageous for particulates, and the surface tension of the water may be decreased, which reduces the start voltage of the electrostatic spray, so that the spray efficiency of the nozzle 112 may be improved.

A third film 130 may be stacked on the second film 120. The third film 130 may include a third base layer 132 and a flow path FP in the third base layer 132.

The third base layer 132 may be a transparent film. The third base layer 132 may include polyimide, acrylic, polycarbonate, polyethylene terephthalate, polyethylene, polypropylene, polysulfone, polymethyl methacrylate, triacetylcellulose, polydimethylsiloxane, etc.

The flow path FP may be a passage through which water H₂O, which is to be electrostatically sprayed, may flow, and may include a groove formed on the surface of the third base layer 132 facing the second film 120. In another example embodiment, the flow path may be formed inside the third base layer 132. The flow path FP may have a shape, such as a mesh pattern, in which the plurality of nozzles 112 are connected.

The water H₂O flowing through the flow path FP of the third film 130 may be supplied to the nozzle 112 and sprayed from the nozzle 112 when the voltage is applied for the electrostatic spray. To this end, the second film 120 may include a plurality of second openings OP2 that are arranged at positions overlapping the plurality of nozzles 112. The plurality of second openings OP2 connect the plurality of nozzles 112 to the flow path FP. In this case, a width of the second opening OP2 may be greater than that of the first opening OP1 that is provided at the center of the nozzle 112, and thus the flow rate of the water which has flown out through the first opening OP1 may be increased. In addition, the plurality of surrounding electrodes 122 may be arranged in such a configuration that a plurality of second openings OP2 are surrounded by the plurality of surrounding electrodes 122, respectively, and thus the water H₂O may be heated when passing through the second openings OP2.

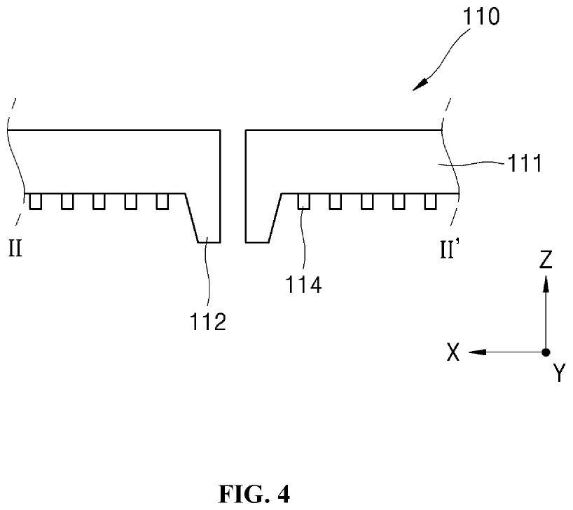

FIG. 4 is a cross-sectional view schematically illustrating another example of the cross section cut along I-I′ in FIG. 2.

FIG. 4 illustrates only the first film 110 for convenience of description. Referring to FIG. 4, a plurality of protrusions 114 may be further provided on the surface of the first base layer 111. The plurality of protrusions 114 may be positioned around the nozzle 112.

The plurality of protrusions 114 may be shorter than the nozzle 112. For example, the plurality of protrusions 114 may have a height of 20 μm or less. In addition, the plurality of protrusions 114 may have hydrophobicity. The protrusions 114 may prevent the droplets from forming into drops on the nozzle 112, to thereby improve the efficiency of electrostatic spray.

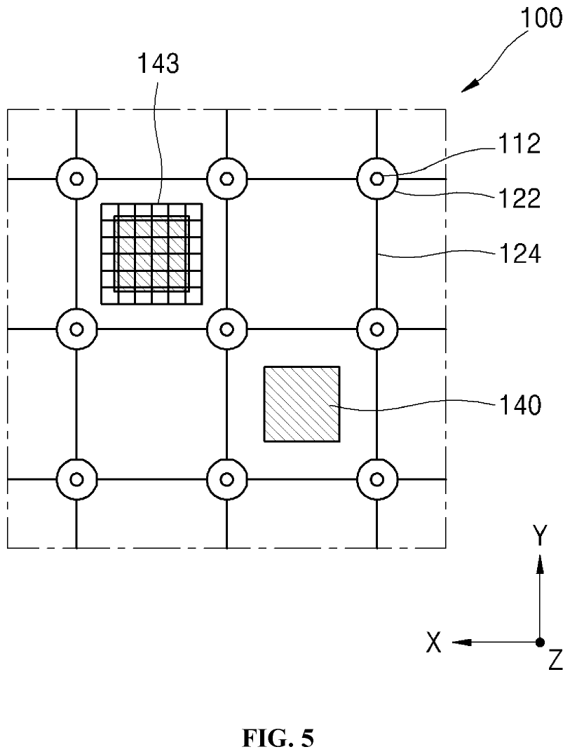

FIG. 5 is a plan view illustrating a modified example of the electrostatic spray nozzle film shown in FIG. 1.

FIG. 5 shows a bottom surface of the electrostatic spray nozzle film 100. Referring to FIG. 5, as described above, the plurality of nozzles 112 may be arranged in such a configuration that the nozzles 112 are spaced apart from each other, and each surrounding electrode 122 may be positioned to surround each nozzle 112 in a plan view. In addition, the plurality of surrounding electrodes 122 may be electrically connected to each other by the connection wiring 124.

In addition, the electrostatic spray nozzle film 100 may further include a plurality of light-emitting devices 140. The light-emitting device 140 may include, for example, a light-emitting diode. The light-emitting device 140 may emit various lights according to an environment in which the electrostatic spray nozzle film 100 is used.

For example, when the electrostatic spray nozzle film 100 is used in a plant cultivation device for growing plants, the light-emitting device 140 may supply light suitable for plant growth. In addition, some of the plurality of light-emitting devices 140 may emit ultraviolet light, and a discharge net 143 or a discharge electrode may be installed around the light-emitting device 140 emitting the ultraviolet light. Therefore, pests may be induced by ultraviolet rays and the pests may be eradicated by the discharge net 143 or the discharge electrode. For example, the discharge net 143 or the discharge electrode may be shaped into a cover for covering the light-emitting device 140 for emitting ultraviolet rays.

In another example embodiment, when the electrostatic spray nozzle film 100 is used in a sterilization device, the light-emitting device 140 may emit ultraviolet light.

The light-emitting device 140 may be positioned on a lower surface of the electrostatic spray nozzle film 100. For example, a flexible printed circuit board (FPCB) may be positioned on the lower surface of the electrostatic spray nozzle film 100, and the light-emitting device 140 may be mounted on the FPCB. In this case, the protrusion 114 described in FIG. 4 may not be provided at a location where the FPCB or the light-emitting device 140 is positioned.

In another example embodiment, the light-emitting device 140 may be positioned on an upper surface of the electrostatic spray nozzle film 100. Since the first base layer 111 in FIG. 3, the second base layer 121 in FIG. 3, and the third base layer 131 in FIG. 3 described above may be formed of transparent films, light generated from the light-emitting device 140 may pass through the electrostatic spray nozzle film 100 to reach a plant or a sterilization target although the light-emitting device 140 is positioned on the upper surface of the electrostatic spray nozzle film 100.

Particularly, the light-emitting device 140 may be arranged at a position that does not overlap with the surrounding electrodes 122 so as to minimize the effect of heat generated from the surrounding electrode 122 and the connection wiring 124. For example, a single light-emitting device 140 may be positioned in an area defined by four surrounding electrodes 122 and a connection wiring 124 connecting the four surrounding electrodes 122.

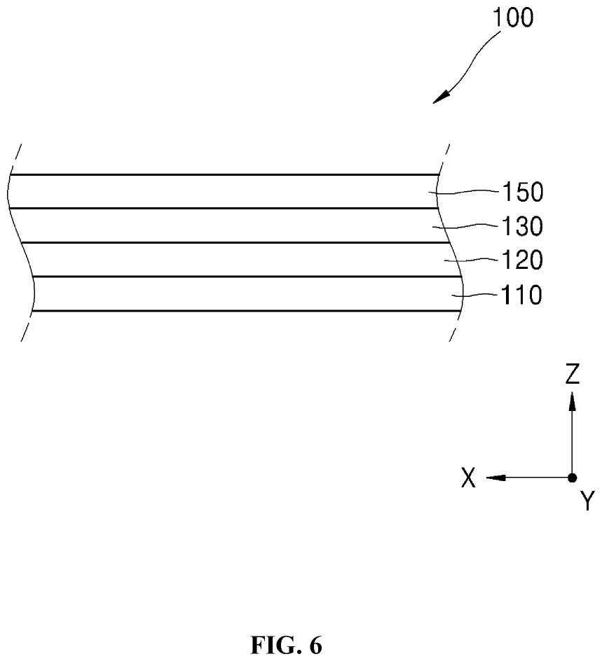

FIG. 6 is a cross-sectional view illustrating another modified example of the electrostatic spray nozzle film shown in FIG. 1.

FIG. 6 schematically shows a cross section of the electrostatic spray nozzle film 100. Referring to FIG. 6, the electrostatic spray nozzle film 100 may include the first film 110, the second film 120, the third film 130, and a fourth film 150 that are sequentially stacked.

The first film 110, the second film 120 and the third film 130 are the same structures or configurations as described above, and thus further detailed descriptions on the first film 110, the second film 120 and the third film 130 are not repeated any more.

The fourth film 150 may be a functional film and may include at least one of an electrochromic film and an antenna film.

The electrochromic film is a film whose color changes depending on the amount of electricity applied thereto, and the amount of light passing through the electrochromic film may be freely controlled. Therefore, when the electrostatic spray nozzle film 100 is used in a plant cultivation device for growing plants, the amount of sunlight and the time of exposure to sunlight may be controlled by the electrochromic film depending on the plant to be grown.

The antenna film may include a film-type antenna for transmission and reception therein. As described below, the electrostatic spray system including the electrostatic spray nozzle film 100 may include a control unit, and the user may transmit and receive information to/from the control unit via the antenna film, control the operation of the electrostatic spray system, or transmit and receive information on the electrostatic spray system.

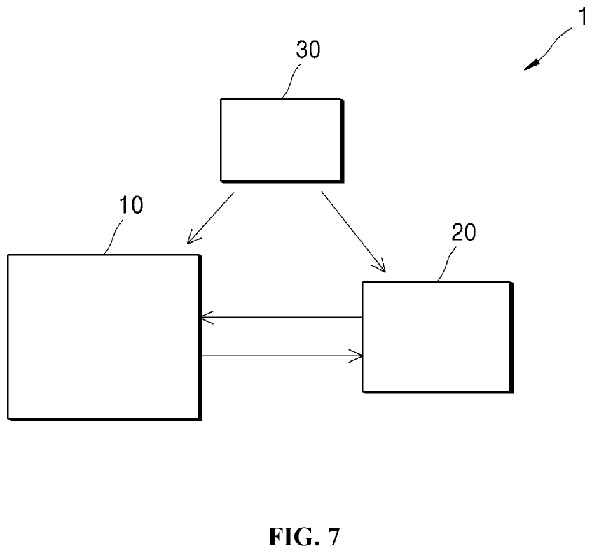

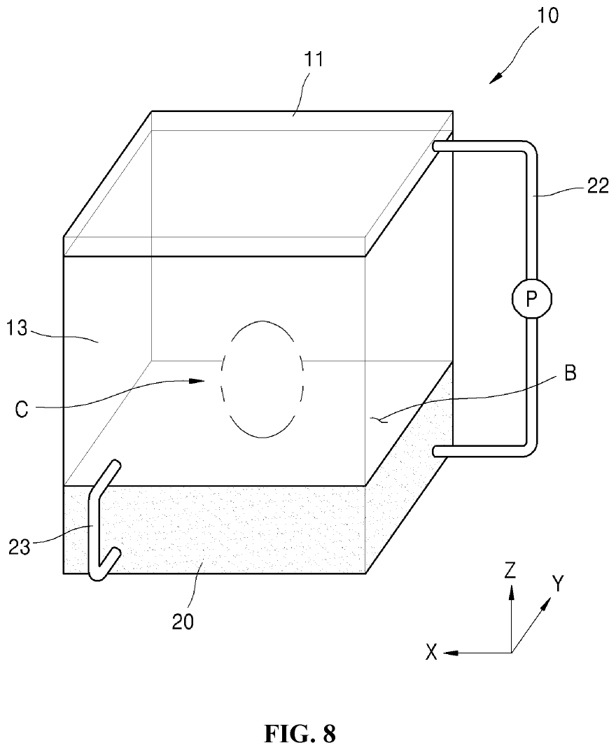

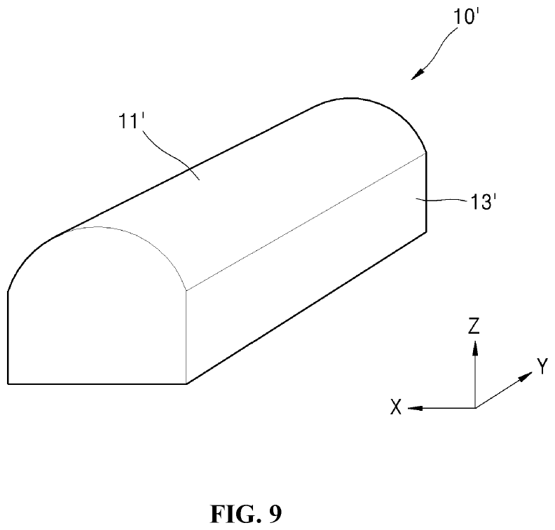

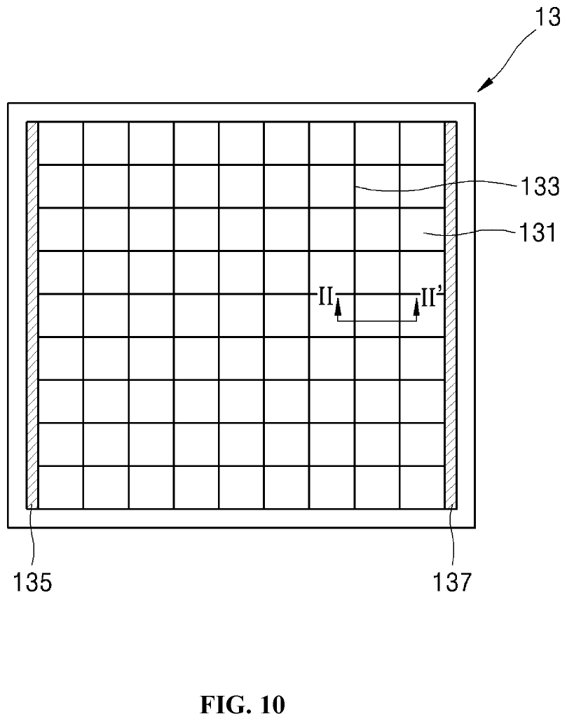

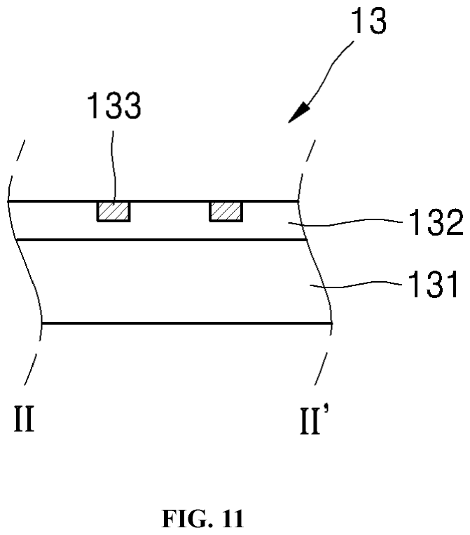

FIG. 7 is a block diagram schematically showing an example embodiment of an electrostatic spray system according to an embodiment of the present disclosure, FIG. 8 is a perspective view schematically illustrating an example embodiment of an electrostatic spray unit and an electric power unit shown in FIG. 7, FIG. 9 is a perspective view schematically illustrating another example embodiment of the electrostatic spray unit shown in FIG. 7, FIG. 10 is a plan view schematically showing an example embodiment of a wall portion shown in FIG. 8, and FIG. 11 is a cross-sectional view schematically illustrating an example of a cross-section cut along line II-II′ in FIG. 10.

At first, referring to FIGS. 7 and 8, an electrostatic spray system 1 according to an embodiment of the present disclosure may include an electrostatic spray unit 10 for electrostatically spraying water, an electric power unit 20 for supplying power and water to the electrostatic spray unit 10, and a control unit 30 for controlling operations of the electrostatic spray unit 10 and the electric power unit 20.

Although the electrostatic spray unit 10 is illustrated as a substantially hexahedral shape and the electric power unit 20 is illustrated to be positioned under the electrostatic spray unit 10 in FIG. 8, the present disclosure is not limited thereto, and the position of the electric power unit 20 may be variously changed. In addition, as shown in FIG. 9, the wall portion 13′ and the cover portion 11′ of the electrostatic spray unit 10′ may have various shapes such as a vinyl greenhouse shape.

Referring to FIG. 8 again, the electrostatic spray unit 10 may include an inner space in which a target C for electrostatically spraying with water is positioned and defined by a wall portion 13 together with a floor B, and a cover 11 positioned on the wall portion 13 and covering the inner space.

At least one of the cover 11 and the wall portion 13 may include the electrostatic spray nozzle film 100 in FIG. 1, and water may be electrostatically sprayed into the inner space of the electrostatic spray unit 10. In addition, an entrance, which is selectively opened or closed, may be provided with at least one of the wall portion 13 and the cover 11, and thus the target C for electrostatically spraying with water may be inserted into or withdrawn from the electrostatic spray unit 10.

In addition, the electrostatic spray unit 10 may further include a sensor for detecting temperature and humidity of an inside of the electrostatic spray unit 10.

The electric power unit 20 may include a fuel cell. The fuel cell may include a polymer electrolyte fuel cell, an alkaline fuel cell, etc. The fuel cell may include an anode, a cathode, and an electrolyte between the anode and cathode.

Hydrogen, which is a fuel, is oxidized to generate hydrogen ions and electrons at the anode, and the hydrogen ions move to the cathode via the electrolyte while the electrons move to the cathode along the external conductive line, to thereby generate electric energy. Hydrogen ions react with oxygen at the cathode to generate water.

The electric power unit 20 and the electrostatic spray unit 10 may be connected with each other by a first pipe 22, and the water generated from the fuel cell may be supplied to the electrostatic spray unit 10. A pump P is mounted on the first pipe 22, and the water generated from the fuel cell may be easily supplied to the electrostatic spray unit 10. Particularly, the first pipe 22 and the pump P need be made of an insulating material for applying a high voltage to the water.

For example, when the cover 11 includes the electrostatic spray nozzle film 100 in FIG. 1, the first pipe 22 is communicated with the flow path FP in FIG. 3 of the electrostatic spray nozzle film 100 in FIG. 1 in such a configuration that the water generated from the fuel cell is supplied to the cover 11 and the supplied water is electrostatically sprayed into the inner space of the electrostatic spray unit 10 by the electrostatic spray nozzle film 100 in FIG. 1.

Particularly, as the water H₂O discharged from the fuel cell is ultrapure water and has low electrical conductivity, the water H₂O discharged from the fuel cell may be advantageous for electrostatic spraying, and as the water H₂O discharged from the fuel cell does not include foreign substances or ions, the problems regarding the nozzle 112 in FIG. 3, such as a nozzle clogging, may be prevented.

In addition, when the electrostatic spray nozzle film 100 in FIG. 1 includes the light-emitting device 140 in FIG. 5, an electrochromic film, etc., the electric energy generated from the fuel cell may be used for overall operations of the electrostatic spray unit 10, for example, for operating the light-emitting device 140 or the electrochromic film and applying a voltage to water for the electrostatic spray.

In addition, the inner space of the electrostatic spray unit 10 and the electric power unit 20 may be connected to each other by a second pipe 23. Although not shown in the drawings, the second pipe 23 may include a valve, a pump, etc. The second pipe 23 may supply the humidified air in the electrostatic spray unit 10 to the fuel cell to prevent the efficiency reduction of the fuel cell in the electric power unit 20.

For example, when the fuel cell is a polymer electrolyte fuel cell and the polymer film is dried, the conductivity of the hydrogen ion decreases and the polymer film is contracted, and as a result, the contact resistance increases between the electrode and the polymer film, and the efficiency of the fuel cell is drastically reduced. However, when the humidified air in the inner space of the electrostatic spray unit 10 is continuously supplied to the electric power unit 20 through the second pipe 23, the efficiency reduction of the fuel cell caused by the above-described problem may be prevented.

That is, according to the present disclosure, the electric power and water generated from the fuel cell of the electric power unit 20 may be transferred to the electrostatic spray unit 10, and the humidified air in the electrostatic spray unit 10 may be transferred back to the electric power unit 20, to improve the efficiency of the fuel cell.

The control unit 30 may control overall operations of the electrostatic spray unit 10 and the control unit 20. For example, for performing the electrostatic spray, the control unit 30 may drive the pump P to supply water to the electrostatic spray unit 10 and may apply a constant voltage to the supplied water and the surrounding electrodes 122 in FIG. 3. In addition, the control unit 30 may control the spraying amount and time, or the operation of the light-emitting device 140 in FIG. 5 and/or the electrochromic film in the electrostatic spray unit 10 according to the temperature and humidity of the inner space of the electrostatic spray unit 10 detected from the sensor in the electrostatic spray unit 10.

In addition, the control unit 30 may communicate with a terminal of a user by the antenna of the electrostatic spray unit 10, transmit a state of the electrostatic spray system 1 to the user, or receive a command for an operation of the electrostatic spray system 1 from the user.

The electrostatic spray system 1 described above may be applied to various fields.

For example, the electrostatic spray system 1 may be used as a sterilizer for sterilizing and disinfecting the target C by using the electrostatic spray. In such a case, the target C for the electrostatic spray may include items that require hygiene management, such as articles used jointly by several people, baby items, mobile phones, and tableware, etc.

When the electrostatic spray system 1 is used as the sterilizer, the electrostatic spray nozzle film 100 in FIG. 3 and the light-emitting device for emitting ultraviolet rays 140 in FIG. 5 may be provided with at least one of the wall portion 13 and the cover 11.

For example, the wall portion 13 and the cover 11 may have the same structures. For example, both of the wall portion 13 and the cover 11 may include the electrostatic spray nozzle film 100 in FIG. 3 and the light-emitting device 140 in FIG. 5 for emitting ultraviolet light.

On the other hand, since having a feature of straightness, the ultraviolet light generated from the light-emitting device 140 in FIG. 5 has difficulties in reaching the curved or shaded portion of the target C, and thus the portion of the target C where the ultraviolet light does not reach is not sterilized by the ultraviolet light, or the sterilization effect is deteriorated. However, when water is electrostatically sprayed from the wall portion 13 and the cover 11, the sterilization and disinfection performance may be improved as water droplets having sterilization efficacy can reach the portion of the target C where the ultraviolet light does not reach.

Specifically, when water H₂O is electrostatically sprayed, the water may be ionized to the fine water droplets having strongly reactive negative ions, such as hydroxyl radical and superoxide radical, and the fine water droplets may be sprayed. Thus, as having high reducibility, the negative ions strongly react with positive hydrogen ions in microbial membranes or viruses existing on the surface of the target C and are reduced to water. Accordingly, the microbes and viruses may be inactivated by those processes.

That is, according to the present disclosure, the shaded position of the target C where the ultraviolet rays does not reach may be sterilized just by electrostatically spraying water droplets having sterilization efficacy from the wall portion 13 and the cover 11, and thus the sterilization and disinfection effect of the electrostatic spray system 1 may be improved.

Assuming that the power consumption of the electrostatic spray unit 10 is about 2 kW per hour and the energy efficiency of the fuel cell is 60%, the hydrogen required for the fuel cell is about 0.084 kg, and the amount of water generated at this time is about 0.76 kg (0.76 L). That is, since a sufficient amount of water required for electrostatic spraying can be generated from the fuel cell of the power supply unit 20, the electrostatic spray system 1 according to the present disclosure may not separately supply water for electrostatic spraying from the outside.

In addition, after the sterilization and disinfection of the target C are completed by using the electrostatic spray and the ultraviolet rays, the target C may be dried by the surrounding electrode 112 in FIG. 2. Specifically, after the sterilization and disinfection of the target C is completed, the supply of water H2O to the flow path FP in FIG. 3 may be stopped by the pump P, and the inside of the electrostatic spray unit 10 may be heated by the surrounding electrode 112 in FIG. 2 to which a voltage is applied, to thereby dry the target C.

In another example embodiment, the electrostatic spray system 1 may be a plant cultivation device where the target C of electrostatic spray is a plant. The plant may include, for example, crops or edible plants such as vegetables, fruits, and grains, etc. Particularly, plants may have different cultivation environments depending on the type thereof, but the electrostatic spray unit 10 may provide an environment suitable for the cultivated plant.

To this end, the cover 11 may include the electrostatic spray nozzle film 100 in FIG. 3 and spray water into the inner space of the electrostatic spray unit 10.

The water may be generated from the fuel cell of the electric power unit 20 and be supplied to the electrostatic spray unit 10.

In general, plant cultivation requires about 0.05 L/h of water per 3.3 m². As described above, assuming that the power consumption of the electrostatic spray unit 10 is about 2 kW per hour and the energy efficiency of the fuel cell is 60%, the hydrogen required for the fuel cell is about 0.084 kg and the amount of generated water may be about 0.76 kg (0.76 L). That is, under the conditions, an amount of water capable of growing plants is generated in the electrostatic spray unit 10 having a width of approximately 49.5 m² from the power supply unit 20, and thus water may not be supplied from the outside to the electrostatic spray unit 10. That is, according to the present disclosure, power and water generated from the fuel cell of the electric power unit 20 may be supplied to the electrostatic spray unit 10 and may be used for the plant cultivation, and the humidified air in the electrostatic spray unit 10 may be supplied to the electric power unit 20 and may be used for improving the efficiency of the fuel cell of the electric power unit 20.

A bottom B of the electrostatic spray unit 10 may be coated with soil in a certain thickness or more to allow plants to take root, and a drain may be provided with the electrostatic spray unit 10 to discharge the water, which is supplied from the cover 11, to the outside.

Both the cover 11 and the wall portion 13 may be formed transparently, and the plant may be grown by natural lighting. Therefore, the plant may be grown organically in a clean environment without pests and pests by preventing external pests from approaching the plant.

In addition, at least one of the cover 11 and the wall portion 13 may further include a light-emitting device 140 in FIG. 5 for supplying light suitable for plant growth.

In an optional embodiment, some of the light-emitting devices 140 in FIG. 5, which are included in at least one of the cover 11 and the wall portion 13, may emit ultraviolet rays, and the discharge net 143 in FIG. 5 or the discharge electrode may be installed around the light-emitting device 140 in FIG. 5 from which the ultraviolet rays are emitted. Therefore, the pests may be induced by ultraviolet rays and the pests may be eradicated by the discharge net 143 in FIG. 5 or the discharge electrode. For example, the discharge net 143 in FIG. 5 or the discharge electrode may be shaped into a cover for covering the light-emitting device 140 in FIG. 5 from which the ultraviolet rays are emitted.

In addition, the cover 11 and the wall portion 13 may further include a fourth film 150 in FIG. 6 that is a functional film. The fourth film 150 in FIG. 6 may include at least one of an electrochromic film and an antenna film. The electrochromic film is a film whose color changes depending on the amount of electricity applied thereto, and the amount of light passing through the electrochromic film may be freely controlled. Therefore, the amount of sunlight and the time of exposure to sunlight may be controlled by the electrochromic film depending on the plant to be grown.

In addition, as shown in FIG. 10, the wall portion 13 may include a heater electrode 133 to adjust the temperature of the inner space of the electrostatic spray unit 10. Furthermore, the surrounding electrode 122 in FIG. 2 and the connection wiring 124 in FIG. 2 may also adjust the temperature of the inner space of the electrostatic spray unit 10.

Referring to FIGS. 10 and 11, the wall portion 13 may include a support 131 and a base layer 132 on the support 131.

The support 131 may be made of a material that is transparent, has excellent insulation properties, and has high rigidity enough to maintain the shape of the wall portion 13. For example, the support 131 may be formed of various materials, such as a glass material or a plastic material such as polyethylene terephthalate (PET), polyethylene naphthalate (PEN), and polyimide.

The base layer 132 may be positioned on a surface of the support 131, and the heating electrode 133 may be positioned on an opposite surface of the base layer 132.

The base layer 132 may include a transparent film. The base layer 132 may be made of polyimide, acrylic, polycarbonate, polyethylene terephthalate, polyethylene, polypropylene, polysulfone, polymethyl methacrylate, triacetyl cellulose, polydimethylsiloxane, etc.

The heating electrode 133 may be inserted from a surface of the base layer 132 in a direction of the thickness of the base layer 132, and be positioned in the base layer 132. A plurality of grooves may be formed on the base layer 132 at positions where the heating electrodes 133 are formed, and a conductive paste may be printed in the grooves by a screen printing, to thereby form the heating electrodes 133 on the base layer 132. The conductive paste may include a mixture of a conductive particle, a binder, and an organic vehicle with a solvent. For example, the conductive particle of the conductive paste include silver, palladium, platinum, copper, and the like, and the binder may include a lead oxide (PbO)-boric oxide (B2O3)-silicon oxide (SiO2) based inorganic binder or a lead oxide (PbO)-bismuth oxide (Bi2O3)-silicon oxide (SiO2) based inorganic binder. In addition, the organic vehicle may include a cellulose-based or acrylic resin, and the solvent may include terpineol, butyl cabitol acetate (BCA), and the like. However, the present disclosure is not limited thereto, and various materials may be used as the conductive paste.

In another example embodiment, the heating electrode 133 may be formed in the grooves of the base layer 132 by a deposition method or the like. At this time, a seed layer may be formed in the grooves of the base layer 132 by the screen printing or the like in advance so as to facilitate the deposition of the heating electrode 133.

The heating electrode 133 may have a lattice pattern, and a first terminal 135 and a second terminal 137, which are connected to the electric power unit 20 in FIG. 7, are connected to both ends of the heating electrode 133, and the temperature of the inner space of the electrostatic spray unit 10 may be adjusted by supplying power, to thereby prevent condensation in winter.

Although the heating electrode 133 is inserted into the surface of the base layer 132, the support 131 to which the base layer 132 is attached may have excellent insulation properties, and thus the heating electrode 133 may be formed to be inserted into the surface of the base layer 132 facing the inner space of the electrostatic spray unit 10. At this time, since the inner space of the electrostatic spray unit 10 is in a humidified state by the electrostatic spraying, the wall portion 13 may further include a protection film for covering the heating electrode 133 so as to prevent corrosion of the heating electrode 133.

For example, the heating electrode 133 may be formed to have a line width of 0.1 um to 50 um. Therefore, the light transmittance of the base layer 132 may not be hindered by the heating electrode 133, and the wall portion 13 may maintain a transparent property as a whole.

In addition, the fourth film 150 in FIG. 6 shown and described in FIG. 6 may be further positioned on the wall portion 13. The fourth film 150 in FIG. 6 is a functional film and may include at least one of an electrochromic film and an antenna film.

Although the present disclosure has been described with reference to an embodiment shown in the drawings, this is merely an example, and it will be understood by a person skilled in the art that various modifications and embodiments may be allowable therefrom. Therefore, the true technical protection scope of the present disclosure should be determined by the technical spirit of the appended claims.

1. An electrostatic spray nozzle film comprising:
a first film including a first base layer and a plurality of nozzles in such a configuration that each of the plurality of nozzles protrudes outward from a surface of the first base layer, has a truncated column shape, and has a first opening therein penetrating through the first base layer;
a second film including a second base layer positioned on the first film and a plurality of surrounding electrodes positioned on the second base layer and corresponding to the nozzles, respectively; and
a third film positioned on the second film and having a flow path through which water for an electrostatic spray flows,
wherein in a plain view, the plurality of surrounding electrodes are arranged to surround the nozzles, respectively, and a same voltage is applied to the plurality of surrounding electrodes.

2. The electrostatic spray nozzle film of claim 1, wherein
the second base layer includes a plurality of second openings connecting the first openings of the plurality of nozzles and the flow path with each other, and
a width of each of the second openings is greater than a width of the first opening corresponding to the second opening.

3. The electrostatic spray nozzle film of claim 2, wherein
the plurality of surrounding electrodes surround the second openings, respectively.

4. The electrostatic spray nozzle film of claim 1, further comprising a light-emitting device between the plurality of surrounding electrodes in a plan view.

5. The electrostatic spray nozzle film of claim 1, further comprising
a fourth film stacked on the third film and including at least one of an electrochromic film and an antenna film.

6. An electrostatic spray system comprising:
an electrostatic spray unit including the electrostatic spray nozzle film of claim 1;
an electric power unit configured to supply power and ultrapure water to the electrostatic spray unit; and
a control unit configured to control operations of the electrostatic spray unit and the electric power unit,
wherein a humidified air in the electrostatic spray unit is supplied to a fuel cell of the electric power unit.

7. An electrostatic spray system comprising:
an electrostatic spray unit including the electrostatic spray nozzle film of claim 2;
an electric power unit configured to supply power and ultrapure water to the electrostatic spray unit; and
a control unit configured to control operations of the electrostatic spray unit and the electric power unit,
wherein a humidified air in the electrostatic spray unit is supplied to a fuel cell of the electric power unit.

8. An electrostatic spray system comprising:
an electrostatic spray unit including the electrostatic spray nozzle film of claim 3;
an electric power unit configured to supply power and ultrapure water to the electrostatic spray unit; and
a control unit configured to control operations of the electrostatic spray unit and the electric power unit,
wherein a humidified air in the electrostatic spray unit is supplied to a fuel cell of the electric power unit.

9. An electrostatic spray system comprising:
an electrostatic spray unit including the electrostatic spray nozzle film of claim 4;
an electric power unit configured to supply power and ultrapure water to the electrostatic spray unit; and
a control unit configured to control operations of the electrostatic spray unit and the electric power unit,
wherein a humidified air in the electrostatic spray unit is supplied to a fuel cell of the electric power unit.

10. An electrostatic spray system comprising:
an electrostatic spray unit including the electrostatic spray nozzle film of claim 5;
an electric power unit configured to supply power and ultrapure water to the electrostatic spray unit; and
a control unit configured to control operations of the electrostatic spray unit and the electric power unit,
wherein a humidified air in the electrostatic spray unit is supplied to a fuel cell of the electric power unit.
